(19) United States
(12) Patent Application Publication
(10) Pub. No.: US 2009/0246795 A1
(43) Pub. Date: Oct. 1, 2009

(54) IMMUNOASSAY DEVICE AND METHOD

(75) Inventors: Yoko Hayashi, Kyoto (JP); Osamu Takehiro, Aichi (JP)

Correspondence Address: GLOBAL IP COUNSELORS, LLP, 1233 20TH STREET, NW, SUITE 700, WASHINGTON, DC, 20036-2680, US

(73) Assignee: Rohm Co., Ltd., Kyoto-shi, Kyoto (JP)

(21) Appl. No.: 12/095,082

(22) PCT Filed: Dec. 21, 2006
(86) PCT No.: PCT/JP2006/325539

§ 371 Date: May 27, 2008

(30) Foreign Application Priority Data
Dec. 22, 2005 (JP) 2005-369096

(51) Int. Cl. G01N 33/53; C12M 1/34

(52) U.S. Cl. 435/7.1; 435/287.2

(57) ABSTRACT

An immunoassay device capable of assaying amount of an antigen by allowing a labeled antibody to specifically bind to a antigen analyte in a sample and assaying a label of a bound product, an interior of a single device has four regions comprises: (1) a first region where the antigen in the sample reacts with a first antibody that is the labeled antibody capable of specifically binding to the antigen, (2) a second region where first antibody that has not bound to the antigen reacts with a second biotin- or avidin-bound antibody, (3) a third region where, depending on whether the second antibody is biotin-bound antibody or avidin-bound antibody, either avidin or biotin is immobilized by immobilization means so as to be unable to move to the fourth region, and the second antibody is captured by the immobilized avidin or biotin, and (4) a fourth region where the label of the first antibody that has bound to the antigen is detected, being constructed in such a way that a solution can move sequentially through each region, the first antibody, which is the labeled antibody such that an antibody component is an F(ab′) fragment or reduced IgG, the F(ab′) fragment or reduced IgG being bound with the label in a predetermined proportion, is included in the first region or an adjacent region, and the second antibody is a biotin- or avidin-bound antibody, being of anti-idiotype antibody against the first antibody and a type that cannot bind to the bound product of the antigen and first antibody, and is included in the second region or an adjacent region.

### TECHNICAL FIELD

The present invention relates to an immunoassay device utilized in fields such as clinical laboratory tests, allowing analytes in a specimen to be quantitatively measured conveniently and rapidly with high sensitivity.

### BACKGROUND ART

Simplified immunoassay devices capable of rapid assay by simple methods of use have been developed as a way to assay substances contained in biological samples such as blood and urine. Recently, the importance and usefulness of such devices have increased with the widespread use of such devices for emergency tests, bedside tests, and the like.

Assay devices based on the principles of immunochromatography, flow-through analysis, immunosensors, and the like are known as such simplified immunoassay devices. The methods employed with these devices are of relatively simple design, being based on the principle of adding a sample and then allowing a solution to move vertically or laterally over a support as an antigen-antibody reaction is brought about, ultimately obtaining a signal that corresponds to the analyte. Signals are commonly detected by labeling antibodies with particles that change color, such as a metal colloid or chromogenic latex, and reading the change in color on a detection surface or detection line. Enzyme reaction-based changes in color and the like are also employed, although the assay operations are complicated.

These simplified immunoassay devices are used for qualitative purposes, basically to determine the presence or absence of an analyte. However, quantification has recently been attempted using these devices as a beneficial diagnosis. These attempts have been based on the fact that the change in color in the detection line after assay is dependent on the quantity (concentration) of analyte. Specifically, ways to assess the change in color in the detection line include measurement with a reflectometer, image analysis with a CCD image sensor (for example, Japanese Laid-open Patent Application No. H8-334511 and No 2000-266751), and methods based on conductivity (for example, Japanese Laid-open Patent Application No. 2001-337065).

The prior art has also disclosed similar immunoassay devices related to methods for capturing the unreacted labeled antibody from the whole labeled antibody and assaying the uncaptured labeled antibody (that is, the reacted labeled antibody). For example, they include U.S. Pat. No. 5,705,338, Japanese Laid-open Patent Application No. H4-16745 and No. 2003-262636, and the like. These assay devices lack adequate quantitative reliability, however. In similar prior art, the use of anti-idiotype antibodies has also been disclosed in Japanese Laid-open Patent Application H7-151757.

A technique referred to as lab-on-chip has recently attracted attention. This is a technique in which substances are mixed, reacted, separated, measured, detected, and the like on a substrate (biochip) of several centimeters referred to as a clinical analysis chip, environmental analysis chip, genetic analysis chip (DNA chip), protein analysis chip (proteome chip), sugar chain chip, chromatograph chip, cellular analysis chip, drug screening chip, or the like depending on the type of subject under analysis. The method of immunoassay used various antigen-antibody reactions on the chip has been disclosed in Japanese Laid-open Patent Application No. 2001-4628.

The above efforts at quantification were undertaken to explore applications with attempts at quantification through mechanical measurement of the results of detection obtained from qualitative devices. There were thus subtle differences in intensity or color variation, with a narrow quantification range, making it difficult to conclude that adequate quantitative reliability would be ensured. The use of enzyme reactions for detection is effective in terms of quantification and greater sensitivity, but problems include the need for cleaning and the addition of reagents, and the like, as well as increased background.

Another problem with the microchips described above is that the measurement is time-consuming because of the need for cleaning and the need for complicated steps such as bringing about a reaction by supplying numerous solutions to the site where the reaction is to take place. Another problem is that multiple injection and introduction regions are also needed and occupy a large portion of the chip surface area, precluding smaller and simpler microchips.

In view of the foregoing, an object of the present invention is to provide an immunoassay device that has the advantages of simplified immunoassay devices, specifically, that is easy to use and is capable of rapid assay in a single step, etc., yet can be used for a variety of antigens and is capable of reliable and rapid quantitative analysis.

### Means for Solving the Abovementioned Problems

As a result of extensive research, the inventors perfected the present invention upon finding that it is possible to implement immunoassay permitting rapid and reliable quantitative analysis by constructing a device that has four different regions inside.

That is, the present invention is an immunoassay device capable of assaying amount of an antigen by allowing a labeled antibody to specifically bind to a antigen analyte in a sample and assaying a label of a bound product,

an interior of a single device has four regions comprising:

(1) a first region where the antigen in the sample reacts with a first antibody that is the labeled antibody capable of specifically binding to the antigen,

(2) a second region where first antibody that has not bound to the antigen reacts with a second biotin- or avidin-bound antibody,

(3) a third region where, depending on whether the second antibody is biotin-bound antibody or avidin-bound antibody, either avidin or biotin is immobilized by immobilization means so as to be unable to move to the fourth region, and the second antibody is captured by the immobilized avidin or biotin, and

(4) a fourth region where the label of the first antibody that has bound to the antigen is detected,

being constructed in such a way that a solution can move sequentially through each region,

the first antibody, which is the labeled antibody such that an antibody component is an F(ab′) fragment or reduced IgG, the F(ab′) fragment or reduced IgG being bound with the label in a predetermined proportion, is included in the first region or an adjacent region, and

the second antibody is a biotin- or avidin-bound antibody, being of anti-idiotype antibody against the first antibody and a type that cannot bind to the bound product of the antigen and first antibody, and is included in the second region or an adjacent region.

In the present invention, it is preferably that the first antibody is included in an excess amount relative to the analyte, and that the second antibody is also included in an excess amount relative to the first antibody. The first antibody can is labeled, for example, with an enzyme. The first antibody may be retained in a retaining region, which is connected to an adjacent part of the first region.

A reagent for detecting the label can is retained in the fourth region. The reagent may be retained in a substrate-retaining region, which is connected to an adjacent part of the fourth region. Examples of the reagent include an electron transport mediator and/or chromogenic substrates. The substrate-retaining region is a region which allows a part or whole of reagents for detecting the label to be retained beforehand or immediately prior to use.

The immunoassay device of the present invention can be the device in the form of a biochip. In this case, the four regions are each composed of small spaces, the immobilization means is, for example, microparticles, and the third and fourth regions are separated by a channel through which the microparticles serving as the immobilization means do not pass. In the biochip-device, a pair of electrodes is formed in the fourth region, and the analyte can be detected by electrochemically method.

Further, the present invention provides an immunoassay method comprising;

bringing about a reaction between an antigen analyte in a sample and a first antibody that is a labeled antibody specifically binding to the antigen, then

allowing an unreacted first antigen to react with a second biotin- or avidin-bound antigen, subsequently

capturing the second antibody with avidin when the second antibody is biotin-bound antibody or with biotin when the second antibody is avidin-bound antibody, and

detecting a uncaptured bound product of the antigen and the first antibody,

wherein the first antibody is a labeled antibody such that an antibody component is an F(ab′) fragment or reduced IgG, the F(ab′) fragment or reduced IgG being bound with a label in a predetermined proportion, and

the second antibody is an anti-idiotype antibody against the first antibody, being of a type that cannot bind to the bound product of the antigen and first antibody.

In the immunoassay method of the present invention, it is also preferably that the first antibody is used in an excess amount relative to the analyte, and that the second antibody is used in an excess amount relative to the first antibody. Moreover, the detection of the analyte can be performed by electrochemically or optically.

### EFFECT OF THE INVENTION

The immunoassay device of the invention enables rapid and highly sensitive quantitative measurement by simple operations. It particularly allows more rapid assay through the binding properties of biotin and avidin. It can therefore be used for emergency testing and bedside diagnosis, and would be extremely useful in the healthcare field. It can also be readily adapted for a variety of antigen analytes, making it highly versatile.

### BRIEF DESCRIPTION OF THE DRAWINGS

FIG. 1 A plan view of an embodiment of a device in the form of a test strip of the invention.

FIG. 2 A plan view of another embodiment of a device in the form of a test strip of the invention.

FIG. 3 A plan and cross views of an embodiment of a device in the form of a biochip of the invention.

FIG. 4 A plan and cross views of another embodiment of a device in the form of a biochip of the invention.

FIG. 5 A plan and cross views of still another embodiment of a device in the form of a biochip of the invention.

FIG. 6 A calibration curve showing a relationship of reflectivity and the human CRP concentration obtained in Test Example 1.

FIG. 7 A calibration curve showing a relationship of rate of absorbance change and the Human CRP concentration obtained in Test Example 2.

FIG. 8 A schematic diagram illustrating the assay principle of the invention.

### LEGENDS

1 first region
2 second region
3 third region
4 fourth region
5 device
6 support
11 antigen
12 first antigen (labeled antigen)
13 second antigen
14 avidin when the second antibody is biotin-bound anti-idiotype antibody or biotin when the second antibody is avidin-bound anti-idiotype antibody
15 biotin when the second antibody is biotin-bound anti-idiotype antibody or avidin when the second antibody is avidin-bound anti-idiotype antibody
31 micropassage
32 channel
33 injection inlet
41 microparticle
42 antibody-retaining region
43 substrate-retaining region
51 electrode

### BEST MODE FOR IMPLEMENTING THE INVENTION

The present invention is described more details below.

The present invention is an immunoassay device in which the amount of antigen can be assayed by allowing a first antibody to specifically bind to an antigen of analyte in a sample and measuring a label of a bound product of the antigen and antibody, wherein the device has the four following regions, being constructed in such a way that solution can move sequentially through each region.

A first region is where an antigen in a sample reacts with a first antibody capable of specifically binding to an antigen. A sample solution can be added directly to the region, or a sample-adding region may be provided upstream, allowing the sample solution to move into the first region. In the first region, after the addition of the sample solution, the first antibody and the bound product of the antigen and the first antibody are not retained but move into a second region. The first antibody comprises any of a variety of labels used in ordinary immunoassay, bound to an F(ab′) fragment or reduced IgG of a monoclonal antibody against the antigen of analyte in the sample, where the F(ab′) fragment or reduced IgG are bound with the label in a given binding ratio, such as 1:1 to 1:n (where n is an integer, preferably 1 to 10), the antibody being included in the first region or an adjacent region. Common methods may also be employed to produce the monoclonal antibody F(ab′) fragments and label the F(ab′) fragments. In addition, unlabeled antibodies and unreacted enzymes mixed in with a final bound product are preferably removed in their entirety through purification.

The use of this type of first antibody in this device is extremely beneficial in improving sensitivity and quantitative reliability. Monoclonal antibodies are commonly used and IgG is usually used in a preparation of the first antibody. Because IgG is usually bivalent binding when unmodified IgG is used as such for the first antibody and is reacted with the antigen analyte, the removal of any that has not reacted in the reaction with the antigen analyte will result in a mixture having a first antibody and antigen binding ratio of 1:1 or 1:2. That is because not the antigen will always bind to the all of binding site in one IgG antibody molecule. If bound product of varying binding ratios passes through the second region, material with a 1:1 ratio will be captured in addition to unreacted material, resulting in a loss of sensitivity and quantitative reliability. By contrast, the present invention involves the use of monovalent binding F(ab′) fragments or IgG half molecules resulting from the reduction of IgG, so that only first antibody and a antigen bound in a binding ratio of 1:1 will remain when an unreacted material is removed, allowing the sensitivity and quantitative reliability to be enhanced.

The first antibody may be held in advance in the first region. An antibody-retaining region may also be provided in a region adjacent to the first region to retain the antibody. The antibody-retaining region can be the same shape, size, and the like as the first region, for example. The antibody-retaining region may have a sample-adding region for adding sample solution, or can function to temporarily retain the sample solution and introduce the sample solution and/or the first antibody into the first region.

Examples of labels that can be used for the first antibody include any commonly used in immunoassay, such as enzymes, dyes, redox substances, coloring particles, magnetic particles, fluorescent substances, luminescent substances, and ferrocene, which can be selected as desired in accordance with the necessary assay sensitivity, etc. When enzymes are used, enzymes that are less susceptible to the effects of substrates and enzymes that are virtually absent in the living body or sample are preferably used. Desirable examples include peroxidase and oxidase enzymes. Examples of oxidase enzymes which can be used include glucose oxidases, pyranose oxidases, alcohol peroxidases, and the like. Examples of other enzymes include dehydrogenase enzymes, but virtually none are absent in the living body or are inexpensively available. The enzymes derived from microbial, bacterial and the like having the same action as enzymes from the living body can be utilized by constructing so that antibodies or the like against such enzymes are included in the first or a second region, ant human enzymes are eliminated. The specificity of enzyme substrates or coenzymes can also be exploited. For example, even though NAD is used as a coenzyme with glucose-6-phosphate dehydrogenase from Leuconostoc mesenteroides, there is no problem if human enzyme may be present in samples because human enzyme is NADP-dependent, and human enzymes will therefore have no action.

Ordinarily, the first antibody is preferably used in an excess amount relative to the antigen in the sample solution, but an assay range can be pre-established to use an amount suited to that range. For example, it will be appropriate to predetermine the measurable antigen concentration or range, and to use an amount 1 to 100 times the concentration or range set with a molar ratio. The conditions for mixing the sample solution and first antibody to bring about a reaction are not particularly limited, and can be determined as desired depending on the type of first antibody, sample solution being assayed, and so forth. The temperature is preferably one, such as about 20 to 40° C., that will not affect the activity of these substances. The time can be set for any device configuration but may range from 30 seconds to about 10 minutes.

The second region is where a reaction is brought about between a second antibody and the first antibody that has not bound with the antigen. In the second region, the solution moving from the first region mixes and reacts with the second antibody, moving as such to a third region. The second antibody is a biotin- or avidin-bound antibody, and is anti-idiotype antibody against the first antibody, being of a type that cannot bind to bound product of antigen and first antibody, and is included in the second region or an adjacent region.

A well known method may be used to allow the biotin to bind to the antibody. When, for example, an amino group of an antibody is used, N-hydroxysuccinimide-biotin may be used. When SH groups (which is obtained by reducing IgG or being digested with pepsin to produce F(ab′) fragments which are further reduced) are used, maleimide-biotin may be used. In such cases, a biotinylation reagent with a spacer may be used to increase the reactivity with avidin. All unlabeled antibody or unreacted biotinylation reagent mixed in with the final bound product is preferably removed by purification.

A well known method may be used to allow avidin to bind to antibodies. For example, avidin with maleimide groups introduced may be allowed to react with the SH groups obtained when IgG is reduced or digested with pepsin to produce F(ab′) fragments which are then further reduced. Commercially available reagents such as 4-(N-maleimidomethyl)cyclohexane-1-carboxylate-N-hydroxysuccinimide ester can be used to introduce maleimide groups into the avidin in such cases. All unlabeled antibody or unreacted avidinylation reagent mixed in with the final bound product is preferably removed by purification.

The anti-idiotype antibodies used here refer to monoclonal antibodies that recognize the structure specific to the antigen-binding site of the antibody component of the first antibody. It is known that there are types of anti-idiotype antibodies that can and cannot inhibit binding between antigen and antibodies thereof. The anti-idiotype antibodies that can be used in the invention are the former, that is, the type that cannot bind to bound product of antigen and first antibody.

The anti-idiotype antibodies can be readily prepared in the usual manner. For example, an anti-idiotype antibody of mouse monoclonal antibody can be prepared by administrating the monoclonal antibody serving as an antigen in the form of a KLH conjugate to mice firstly, and then following the usual methods for producing the monoclonal antibody. A hybridoma that produces anti-idiotype antibodies of the intended type can also be readily obtained by screening with ELISA or the like. The anti-idiotype antibody can therefore be readily mass produced in the same manner as ordinary monoclonal antibodies.

Because virtually all of the unreacted first antibody must be captured, the amount of the second antibody used in the second region in the invention can be an excess amount relative to the amount of the first antibody, more specifically, preferably at least 10 times greater, as calculated in terms of mols. Although it is possible to use an antigen instead of anti-idiotype antibody, the antigen is not used in the device of the invention because the antigen would be limited for practical purposes to an antigen of good stability and an antigen that can be prepared in mass quantities at low cost, thereby limiting the types of an analytes that could be assayed.

The reaction conditions at the second region can be established as desired without any particular limitations. The temperature is preferably one, such as about 20 to 40° C., that will not affect the activity of these substances. The time can be set for any device configuration but may range from 30 seconds to about 10 minutes.

The third region is where, depending on whether the second antibody is biotin-bound antibody or avidin-bound antibody, either avidin or biotin is immobilized by an immobilization means so as to be unable to move to a fourth region, and the second antibody is captured by the immobilized avidin or biotin. As a result of the binding between biotin and avidin in the third region, the second antibody bound to the unreacted first antibody and the unreacted second antibody are captured by the immobilization means and cannot move out of the third region, whereas the bound product of the first antibody and antigen contain no avidin or biotin and thus move into the fourth region without being trapped by the immobilization means. The avidin used in the invention can be used no matter what the source. For example, albumen avidin and actinomycotic streptoavidin can be used.

The immobilization means, which is intended to immobilize avidin or biotin, may be any that does not affect the avidin-biotin reaction, and can be prepared as desired depending on the type of the analyte and the labeled antibody, type of the anti-idiotype antibody, and so forth. It may be a support in the form of filters, woven materials, microparticles, fibers, or the like. Materials that can be used include glass, filter paper, nylon, polystyrene, or the like. Examples of the support in the form of filters, woven materials, and fibers include porous elements such as glass fiber films, porous membranes, filter paper, nylon membranes, or the like. Examples of the support in the form of microparticles include glass beads, polymer beads such as polystyrene, and porous supports such as agarose and chitosan. The avidin or biotin can be immobilized by any well known method such as physical adsorption, covalent bonding, ion bonding, crosslinking, and electrostatic interaction. The immobilization method may not affect the avidin-biotin reaction.

The use of avidin or biotin as the immobilizing substance in the immobilization means, as in the invention, is useful for shortening the reaction time. The effect in shortening the reaction time is particularly pronounced when the immobilization means is in the form of microparticles dispersed in a solution such as buffer. In terms of reaction mode, it is possible to trap the first antibody by allowing the second antibody that has not been labeled with biotin or avid to bind directly to this type of immobilization means, but this will necessitate a reaction time of 10 minutes to about 1 hour. By contrast, it has become apparent that the use of avidin or biotin in the manner of the invention allows the unreacted second antibody and second antibody bound to unreacted first antibody to be captured in 30 seconds to 5 minutes, and in 30 seconds to 1 minute under ideal conditions. This is a major factor involved in shortening the assay time in the present invention.

The fourth region is where the label of the first antibody that has bound to the antigen is detected, and a well-known method of detection can be used as befits the label. The reagent for detecting the label can be included in the fourth region. All of the reagent can be included in the fourth region, or some can be included in the third, second, or first region or other region. It can also be included in a substrate-retaining region that can be provided in a region adjacent to the fourth region. The substrate-retaining region can also be joined, with or without a passage, incrementally or serially to the fourth region, and can be the same shape, size, and the like as the first region. The label can be detected, for example, using a reflectometer, spectrophotometer, or fluorescence detector.

The reagent for detecting the label can be selected as desired according to the label that is used. Examples for when the label is an enzyme include chromogenic substrates, fluorescent substrates, luminescent substrates, organic acids or inorganic acids, or electron transport mediators capable of functioning as electron transport media allowing electrons to be exchanged between the analyte and the electrode, either individually or in combinations of two or more. Combinations of NAD or NADP, enzyme substrates, diaphorase, and chromogenic substrates can be used for dehydrogenase enzymes, for example. Combinations of organic or inorganic acids and chromogenic substrates can be used for peroxidases. Combinations of enzyme substrates, chromogenic substrates and peroxidase can be used for oxidases.

Examples of the chromogenic substrates, fluorescent substrates, luminescent substrates include 1,2-phenylenediamine (OPD), 3,3′,5,5′-tetramethyl benzidine (TMBZ), 2,2′-azynobis-3-ethylbenzothiazoline-6-sulfonic acid (ABTS), 3-(4-hydroxyphenyl) propionic acid (HPPA), tyramine, luminol, luciferin, and the like.

Examples of organic and inorganic acids include hydrogen peroxide, formic acid, acetic acid, and the like. It is also possible to use a reagent that produces them in the device. Glucose and glucose oxidase can be used, for example, as reagents producing hydrogen peroxide in the device. For example, retaining regions can be provided in 2 locations, and the two can be mixed after the start of assay. They can also be retained in other regions.

Examples of the electron transport mediators include alone or as a combination of two or more oxidation-reduction compounds, for example, ferrocene, alkaline metal ferrocyanide (potassium ferrocyanide, lithium ferrocyanide, sodium ferrocyanide, and the like), or alkyl substitution product thereof (methyl, ethyl, propyl substitution product, and the like), methylene blue, phenazine methosulfate, p-benzoquinone, 2,6-dichlorophenol-indophenol, potassium 1,2-naphthoquinone-4-sulfonate, phenazine ethosulfate, viologen, vitamin K. Among these, it is preferably ferrocene, potassium ferrocyanide, and the like.

Methods for retaining such reagents can utilize any suitable well-known method in the art, such as methods in which the reagents as such is dissolved or suspended in a solvent or the like that will not inhibit the reagent function, and is applied and dried, and methods in which the reagent is mixed with or dispersed in a suitable support (such as the immobilization means noted above) and immobilized.

The analyte in the invention indicates analytes in common clinical laboratory tests, and the like, specifically including a variety of components contained in extracts of solids such as stool or tissue, or various components contained in blood, urine, saliva, secretions, or the like. Because only one type of antibody is needed for the analyte in the present invention, the analyte may be a substance having high molecular weight to which several monoclonal antibodies can bind as well as low molecular weight substances (haptens) to which only one monoclonal antibody can bind.

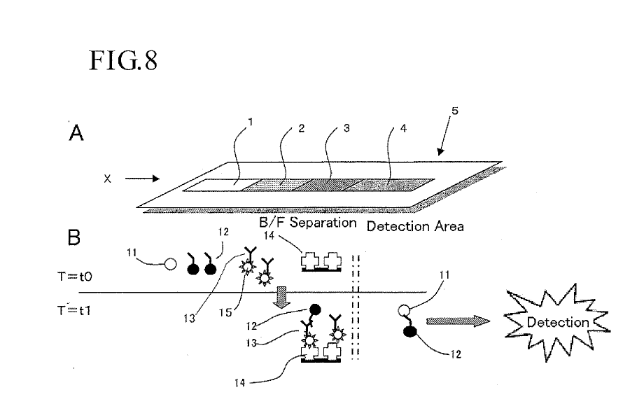

The immunoassay device of the invention was described above. FIG. 8 schematically illustrates the assay principles of the invention. The upper (A) region in the figure is an oblique view of the device 5 as a whole. The arrow X in the figure indicates the direction in which the sample flows. The bottom (B) of the figure illustrates over time the reactions occurring in the device. That is, where at T=t0, the antigen A and first antibody B are mixed in the first region. At T=t1, which is when a suitable period of time has passed, the sample passes through the second region 2 into the third region 3 and fourth region 4. At that time, first antibody B that has not reacted with the antigen A binds to the second biotin-bound antibody C, furthermore binds to the avidin in the third region, and cannot move from the third region 3 to the fourth region 4. Meanwhile, the first antibody B that has reacted with the antigen A passes through the second region 2 and third region 3, arriving in the fourth region 4. In this way, only the first antibody B reacting with the antigen A is detected in the fourth region 4.

No further elaboration of the immunoassay method of the invention will be necessary as it can be readily implemented using the immunoassay device of the invention. The following will be a detailed description of devices in the form of a test strip and a device in the form of a biochip, which may also be referred to as a test piece, as examples of the device of the invention.

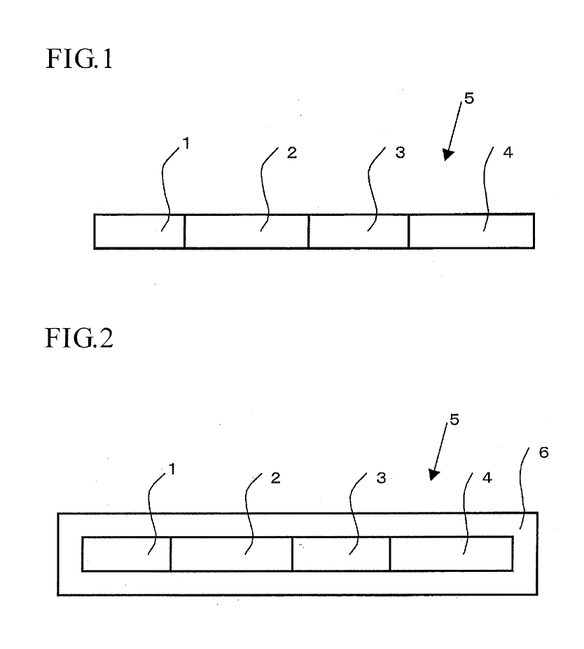

An example of a device in the form of a test strip is first illustrated in FIGS. 1 and 2. The device 5 is composed of a first region 1, second region 2, third region 3, and fourth region 4. These regions can be composed of various materials of various shapes, among which porous supports such as glass fiber membranes, porous membranes, filter paper, and nylon membranes are preferred. The sample solution supplied to the first region or region of it sequentially spreads or is transported through the second region and then the third region to the fourth region. A porous material in the form of a filter, woven material, or fibers is preferably used as the immobilization means in the third region. When the immobilization means is in the form of microparticles, the particle size may be greater than the pore size of the porous support forming the third region. When a sample-adding region is provided upstream of the first region, a blood cell separation membrane or ion exchange membrane can be used, for example. Although each region can be formed on a single porous support, it is also possible to use a support that is ideal for each region. If a different material is used for each region, the materials may be disposed so that the regions are connected to each other, so as not to interfere with the flow of the solution. The size of each region is not particularly limited and must be large enough to achieve the intended purpose, but can be set as desired according to the material type, amount, reaction time, and the like. Examples of combinations of supports for each region include embodiments in which a glass fiber membrane is used for the first region, nitrocellulose membranes are used for the second and third regions, and filter paper is used for the fourth region. Each region is also preferably disposed on a support consisting of a plastic sheet or the like, such as the example of the device in FIG. 2. In FIG. 2, the first region 1, second region 2, third region 3, and fourth region 4 are disposed on a support 6, forming the device 5.

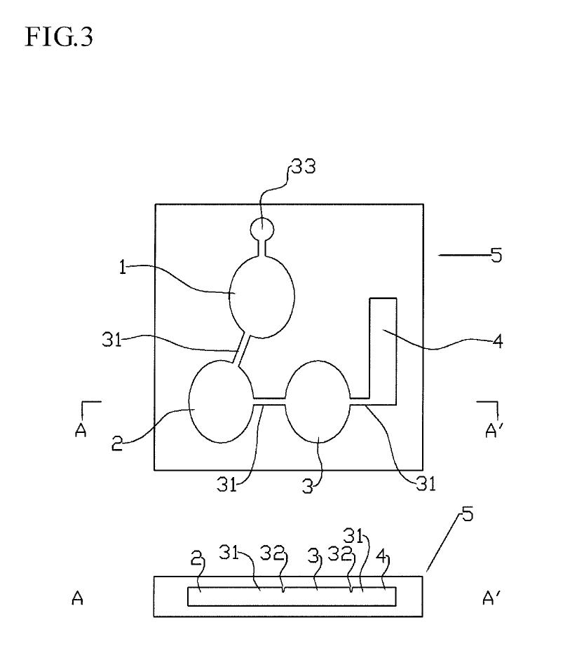

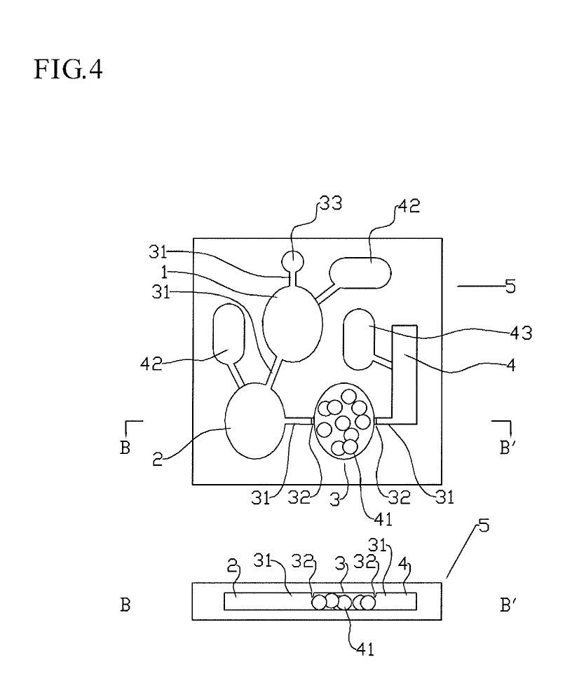

FIGS. 3 and 4 illustrate an example of a device in the form of a biochip. Each of the first region 1, second region 2, third region 3, and fourth region 4 are formed with minute spaces, and are directly joined in series, with or without passages. In FIGS. 3 through 5, they are linked by minute passage 31. The sample solution supplied to the first region or region of it is sequentially transported through the second region and then the third region to the fourth region. Meters with a capacity of 0.5 to 1.5 μL can be provided between each of the first, second, third, and fourth regions to adjust or meter the amount of reaction solution moving to subsequent regions. When the immobilization means comprises microparticles 41, the third region 3 and fourth region 4 are separated by a channel 32 through which the immobilization means cannot pass. A structure similar to the channel 32 is also preferably provided between the second and third regions. Regions for retaining reagents and the like necessary for the reaction (antibody-retaining region 42 and substrate-retaining region 43) can also be provided in regions adjacent to the first, second, and fourth regions. The regions and antibody-retaining and/or substrate-retaining regions are linked in series or incrementally with or without passages. Any substrate-retaining region that is provided can be the same shape, size, and the like as the first region, etc. This will allow reagents to be retained in the substrate-retaining region beforehand or immediately prior to use. The shape, size, and the like of the passages connecting the regions are not particularly limited. For example, the cross section area may be 0.01 μm² to about 100 mm², and the length may be 1 μm to about 100 mm.

The first region is defined as being a space separate from the other regions, the size is preferably about 10⁻² to 10³ mm³. The shape is not particularly limited, provided that it is suitable for the purpose of mixing. The planar and cross section configuration may be any shape, such as polygonal shapes, including tetragonal and trapezoidal shapes, as well as such shapes which are rounded at the corners, and round shapes, and irregular shapes that are asymmetrical on the left and right. An injection inlet 33 for externally injecting the analyte is preferably formed in the first region or an adjacent part.

The first antibody may be held in advance in the first region. When an antibody-retaining region is provided in a region adjacent to the first region, the antibody-retaining region may be formed in addition to or instead of an injection port for injecting the above sample solution. The antibody-retaining region can be the same shape, size, and the like as the first region, for example.

The second region is defined as being a space separate from the other regions, the size is preferably about 10⁻² to 10³ mm³. The second region can be variously shaped in the same way as the first region. The second antibody may also be held in advance in the second region. Alternatively, an antibody-retaining region may be provided in a region adjacent to the second region to retain the second antibody. The antibody-retaining region can be the same shape, size, and the like as the second region, etc.

The third region is defined as being a space separate from the other regions, and includes the immobilization means in the interior. The third region may therefore have enough space to retain the immobilization means, but can be set as desired according to the type, amount, and the like of the immobilization means. It is preferably about 10⁻² to 10³ mm³, for example. The shape can be set a variety of forms and the like, the same as the first or second region.

The immobilization means is housed in the third region, and is preferably in the form of microparticles, preferably with a diameter of 10 μm to about 1 mm.

The channel preferably has a smaller diameter than the diameter of the immobilization means in the third region. Here, “diameter” can be the width, height, length, or the like, depending on the shape of the immobilization means and/or channel, but the “diameter” of the immobilization means is suitably the maximum length (width) per immobilization means unit, and the “diameter” of the channel is suitably the minimum length (width) in the channel cross section. That is, the channel may provides the function or shape that prevents the immobilization means from passing into an outlet (and preferably also an inlet) of the third region in order to ensure that the immobilization means present in the third region stays only in the third region. When the channel is the passage per se, it may be a passage with a diameter narrower than the diameter of the immobilization means, or one or more protrusions may be formed in part or parts of the passage to narrow the diameter in the part or parts.

The fourth region is defined as being a space separate from the other regions, and the size and shape can be set as desired according to the type, amount, and the like of the sample solution, the detection method (procedure), and so forth. Specifically, it can be a variety of shapes of about 10⁻² to 10³ mm³, for example. When the detection method is an optical method, for example, the shape and size of the detection part must ensure a light path of a certain length so as to allow light to be directed onto the product produced from the analyte solution, label, or substrate, and the light to be sensed. When an electrochemical method is used, the fourth region must be formed so that the charge of a solution containing the analyte solution can be sensed and so that a pair of electrodes based on a conductive material is in contact with the solution.

Here, the pair of electrodes can be any material of any size or shape that is ordinarily capable of functioning as an electrode. Examples include single layers, or laminates of two or more layers, of graphite, carbon, carbon black, or the like; metals such as gold, platinum, silver, copper, and aluminum, or alloys thereof, or conductive oxides or the like, such as SnO₂, In₂O₃, WO₃, and TiO₂. The electrodes may be formed by applying pieces of conductive material to the biochip, embedding portions, or employing a printing technique such as screen printing using a conductor paste.

In this way, an electrochemical procedure can be adopted to analyze analytes by a simple method for detecting current, voltage, or the like, without any need to use cumbersome, expensive, large-scale devices such as conventional optical detection devices. The expenses associated with analysis can therefore be reduced, and smaller biochips and smaller, less expensive assay devices can be realized.

The biochip described above can be formed with the same materials as the conventional chips referred to by the various names noted above. Examples include organic compounds such as PET (polyethylene terephthalate), PDMS (polydimethyl siloxane), PMMA (polymethyl methacrylate), PC (polycarbonate), PP (polypropylene), PS (polystyrene), PVC (polyvinyl chloride), polyethylene, polysiloxane, allyl ester resins, cycloolefin polymer and the like, and inorganic compounds such as Zeonor, silicon, quartz, glass, ceramic and the like.

The biochip of the present invention can be readily produced by laminating first and second base plates having patterns in a variety of shapes based on indentations in one or two sides, for example, by means of welding, adhesives, ultrasonic treatment, or the like. Specifically, a mold having a shape corresponding to the part of the desired first to fourth region retaining region, passage, or the like is prepared. The mold can be formed by mechanical machining. A resin is then molded or the like in the mold, giving a base plate onto which the shape of each of part has been transferred. Finally, two base plates are laminated so that the patterns correspond to each other. A base plate with a pattern corresponding to the part of the desired first to fourth region, retaining region, passage, or the like may be on one side, and the other may be a flat base plate. Injection molding, imprinting, or the like may also be used instead of molding with a mold. One or both sides of a flat base plate may directly subjected to a photolithography process, mechanical machining, or the like, giving a base plate on which a pattern corresponding to the part of the desired first to fourth region, retaining region, passage, or the like has been transferred. The biochip of the invention may be handled (such as sample introduction, mixing, stirring, sample transfer, detecting, etc.) manually, automatically, or semi-automatically. For example, stirring and mixing, sample transfer or the like can be done by methods using a pump or methods making use of vibrations or centrifugal force.

### EXAMPLES

The invention is further elaborated in the following examples of the invention, but the invention is not limited to these examples.

### 1. Preparation of Biotin-Labeled Anti-Idiotype Antibodies

### (1) Preparation of Immunogen

3 μL of glutaraldehyde (70%) was added to 2 mL of 0.1 M phosphate buffer (pH 6.7) containing 4 mg KLH (by Merck) and 5 mg of commercially available anti-human CRP mouse monoclonal antibody (by Oriental Yeast), and a reaction was brought about for 1 hour at room temperature. After the reaction, reaction product gives an immunogen by dialyzing with PBS buffer solution.

### (2) Immunization

8-week old BALB/c mice were intraperitoneally administered 200 μL emulsion mixture of 1 mL Freund's complete adjuvant and 1 mL of a 0.5 mg/mL solution of the above immunogen. The animals were then intraperitoneally administered 200 μL of an equivalent emulsion mixture of the above immunogen and Freund's incomplete adjuvant three times at 2-week intervals. 2 weeks after the third time, the animals were intravenously administered 100 μL of the above 0.5 mg/mL immunogen.

### (3) Cellular Fusion of Spleen Cells

After 3 days, spleen cells were extracted from the mice, and were suspended in DMEM media and washed. Meanwhile, the mouse myeloma cell line P3X63Ag8 (by Dainippon Pharmaceutical) was cultured to the log growth phase suitable for cellular fusion, and the cells were collected by centrifugation. Cellular fusion was brought about with the use of PEG (PEG 4000).

### (4) Hybridoma Preparation and Screening

Hybridomas were cultured using 10% FCS-supplemented DMEM media and HAT media, and were screened in the following manner. Primary screening of the hybridoma culture supernatant was done by sandwich ELISA using, as the solid phase antigen, purified F(ab′)₂ fragments digested with pepsin from anti-human CRP mouse monoclonal antibody and using commercially available HRP-labeled goat anti-mouse IgG (Fc) antibody (by ICN) as secondary antibody. For secondary screening, CRP was pre-reacted with the above solid phase antigen, and the same operations were performed to determine types in which binding was inhibited by the CRP. Positive well cloning and screening resulted in the two clones 13D and 14A as hybridomas producing antibodies that specifically recognize anti-human CRP monoclonal antibodies and that undergo inhibited binding due to the CRP.

### (5) Preparation of Antibody

Out of the resulting hybridoma cell lines, 13D was mass cultured to harvest anti-idiotype antibodies. The culture supernatant was purified on a Protein A column (by Amersham Biosciences).

### (6) Preparation of Biotin Label

The antibodies obtained in (5) were labeled with NHS-Biotin (by Pierce) in accordance with the accompanying protocol. Unreacted biotinylation reagent was filtered off by gel filtration.

### 2. Preparation of HRP-Labeled Anti-Human CRP Antibody

### (1) Pepsin Digestion

0.3 mg pepsin (by Sigma Aldrich) and 5 mg anti-human CRP mouse monoclonal antibody were mixed with 1 mL of 0.1 M acetic acid buffer (pH 4.0), and a reaction was brought about for 24 hours at 37° C. Purification by gel filtration following the reaction gave 1.8 mg of F(ab′)₂ fragments. The same commercially available anti-human CRP mouse monoclonal antibody as above was used.

### (2) Reduction

The F(ab′)₂ fragment solution was displaced with 0.1 M phosphate buffer (pH 6.5) containing 5 mM EDTA using an ultrafiltration membrane. A reaction was brought about for 2 hours at 37° C. with the addition of 0.1 M cysteamine solution to the antibody solution. Purification by gel filtration following the reaction gave 1.0 mg F(ab′)₂ fragment.

### (3) Preparation of HRP Maleimide

5 mg horse radish peroxidase (HRP, by Roche Diagnostics) was dissolved in 750 μL of 0.1 M phosphate buffer (pH 7.0). 2 mg of 4-(N-maleimidomethyl)cyclohexane-1-carboxylic acid-N-hydroxysuccinimide ester (Zieben Chemicals Tokyo) was dissolved in 120 μL DMF, and a reaction was brought about for 1 hour at 30° C. with the addition of 75 μL HRP solution. Purification by gel filtration following the reaction gave 2.6 mg HRP maleimide.

### (4) Preparation of Conjugate

1.0 mg F(ab′) fragment and 1.0 mg HRP maleimide were mixed, and a reaction was brought about for 24 hours while refrigerated. The reaction gave 1.2 mg/mL HRP-labeled anti-human CRP antibody solution.

### Example 1

### Preparation of CRP Assay Device in Form of Test Strip

### (1 Preparation of Filter Paper for Label Area (First Region)

10 mg BSA and 10 mg glucose were dissolved in 10 mL PBS buffer. The HRP-labeled anti-human CRP antibody prepared in Example 2 was then diluted 5,000-fold with this solution. Filter paper (by Whatman Japan) was impregnated with the solution and dried, then cut to a size of 12 mm×5 mm.

### (2) Preparation of Filter Paper for Biotin Label Antibody (Second Region)

10 mg BSA and 10 mg sucrose were dissolved in 10 mL PBS buffer. 20 μg/mL solution of the biotin-labeled anti-idiotype antibody prepared in Example 1 was prepared with this solution. Filter paper (by Whatman Japan) was impregnated with the solution and dried, then cut to a size of 10 mm×5 mm.

### (3) Preparation of Membrane for Capturing Area (Third Region)

An avidin 1 mg/mL solution was prepared with PBS. A Hi-Flow membrane (by Millipore) cut to a size of 15 mm×150 mm was impregnated with the solution and dried. It was then allowed to stand in 3 mg/mL casein solution, taken out and dried, and cut to a size of 15 mm×5 mm.

### (4) Preparation of Filter Paper for Detection Area (Fourth Region)

5 mg TMBZ and 30 units of pyranose oxidase were dissolved in 1 mL of PBS. Filter paper (by Whatman Japan) was impregnated with the solution and dried, and then cut to a size of 7 mm×5 mm.

### (5) Test Piece Assembly

Double-sided adhesive tape was applied 25 mm width from the top end on a plastic strip (60 mm×5 mm) serving as the support. The membrane of (3) was then applied and secured, leaving about 5 mm of space at the top end of the plastic strip. The filter paper of (4) was then applied in such a way that about 2 mm overlapped with the top end of the membrane of (3). The filter paper of (2) was then applied and secured in such a way that the bottom end of the membrane of (3) overlapped about 2 mm. The filter paper of (1) was then applied and secured in such a way that the bottom end of the filter paper of (2) overlapped about 2 mm.

### Test Example 1

### Quantitative Assay of CRP Using Device in the Form of Test Strip

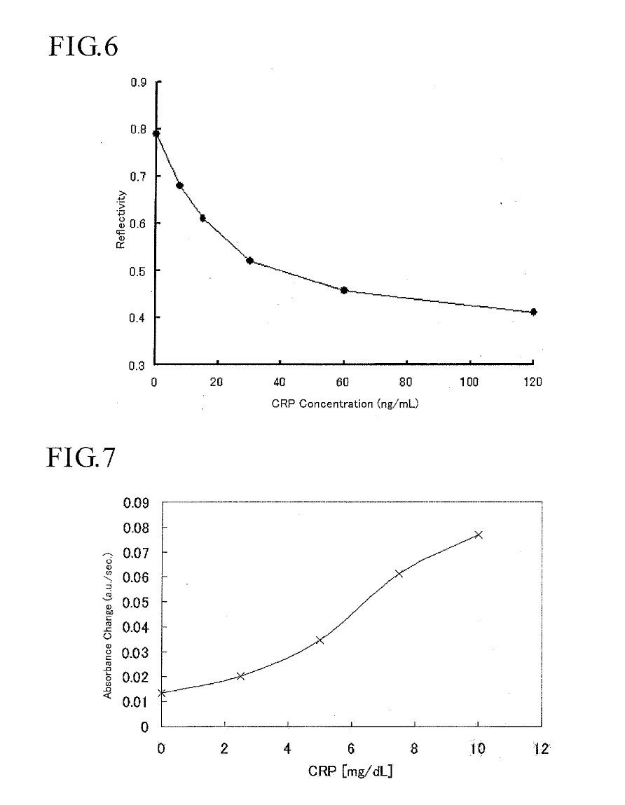

Solutions containing 0, 7.5, 15, 30, 60, or 120 ng/mL CRP were used as specimens. A reaction was started with the addition of 30 μL specimen to the first region of the test strips prepared in Example 3, and after 5 minutes the reflectance at 690 nm was determined with a calorimeter in the fourth region. FIG. 6 gives a calibration curve showing the relation between human CRP concentration and the resulting reflectance. This shows that a good calibration curve from low concentration, revealing that the CRP could be quantified.

### Example 2

### Preparation of CRP Assay Device in the Form of Biochip

A biochip was prepared by laminating together first and second substrates having a pattern such that the first, second, third, and fourth regions were disposed in series by means of recesses on one side, with a channel formed between the third and four regions. Metering regions with a capacity of 0.5 to 1.5 μL were provided between each of the first, second, third, and four regions.

The first region had a space of about 50 mm² (plane area)×1 mm (depth). 10 μL of the 0.05 mg/mL HRP-labeled anti-human CRP antibody prepared in Example 2 was injected as the first antibody.

The second region had a space of about 50 mm² (plane area)×1 mm (depth). 5 uL of the 4.2 mg/mL anti-idiotype antibody was prepared in Example 1 was injected as the second antibody.

The third region had a space of about 50 mm² (plane area)×1 mm (depth). 10 uL of avidin-immobilized sepharose gel (gel with 10 mg/mL bound avidin) was introduced therein. The avidin-immobilized sepharose gel was prepared using NHS activated sepharose gel (by Amersham Biosciences), and the avidin was immobilized on the gel by the method recommended by the supplier.

The fourth region was about 100 mm long, with a cross section area of about 1 mm², for the measurement of absorbance for optical detection. 20 μL SAT-Blue (Dojindo labatories) as the chromogenic reagent was injected.

A channel of about 100 μm in breadth and 200 μm deep, through which no immobilization means could pass, was provided between the third and fourth regions. The first, second, third, and fourth regions were also joined in series by micropassages.

### Test Example 2

### Quantitative Assay of CRP Using Device in the Form of Biochip Prepared in Example 2

Solutions containing 0, 2.5, 5, 7.5, and 10 mg/dL CRP were used as specimens. A reaction was started with the addition of 1.5 μL specimen to the first region of the biochips prepared in Example 2. The solution was then allowed to pass sequentially through the first, second, and third regions. Solutions were metered between the regions by a commonly known method. At this time, the liquid was transported through centrifugal force and was mixed by turbulence resulting from centrifugal force. Lastly, the rate of change in the absorbance at 670 rn was determined in the fourth region. FIG. 7 gives a calibration curve showing the relation between human CRP concentration and the resulting rate of change in the absorbance. This shows that a good calibration curve from low concentration, revealing that the CRP could be quantified with the biochip.

### Example 3

### Preparation of CRP Assay Device in the Form of Biochip Using Electrochemical Detection

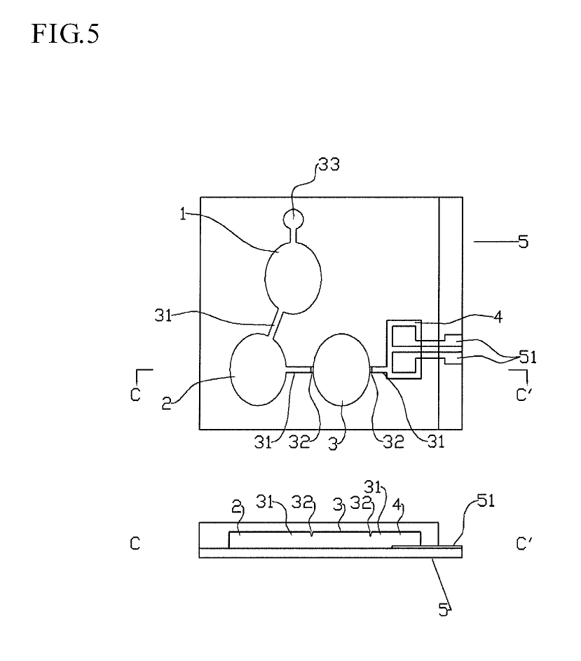

As illustrated in FIG. 5, the biochip in this example had the same structure as the biochip in Example 2, except that a pair of carbon black electrodes 25 was formed in the fourth region, and a redox substance was provided. The electrodes were formed with carbon paste to a length of about 10 mm and a thickness of about 15 μm. The electrodes were disposed at a location corresponding to the fourth region on the bottom substrate forming the biochip. When the top substrate was laminated onto the top of the bottom substrate, a part of the electrodes was located inside the biochip, and a part was exposed. In the fourth region, 3 mM ferrocene and 5 mM hydrogen peroxide were also used as a substrate instead of SAT-Blue.

### Test Example 3

### Quantitative Assay of CRP Using the Device in the Form of the Biochip Prepared in Example 3

1.5 μL specimen containing CRP was added to the biochip, and the solution was transported in sequence through the first, second, and third regions. In the fourth region, the ferrocene was converted to ferricinium ion (FC⁺) by the enzyme label, and this was reduced on the electrodes, allowing the current produced during the redox reaction to be measured. The current level is proportionate to the concentration of the CRP and labeled antibody complex, allowing the CRP to be quantitatively assayed.

1. An immunoassay device capable of assaying amount of an antigen by allowing a labeled antibody to specifically bind to a antigen analyte in a sample and assaying a label of a bound product,
an interior of a single device has four regions comprising:
(1) a first region where the antigen in the sample reacts with a first antibody that is the labeled antibody capable of specifically binding to the antigen,
(2) a second region where first antibody that has not bound to the antigen reacts with a second biotin- or avidin-bound antibody,
(3) a third region where, depending on whether the second antibody is biotin-bound antibody or avidin-bound antibody, either avidin or biotin is immobilized by immobilization means so as to be unable to move to the fourth region, and the second antibody is captured by the immobilized avidin or biotin, and
(4) a fourth region where the label of the first antibody that has bound to the antigen is detected,
being constructed in such a way that a solution can move sequentially through each region,
the first antibody, which is the labeled antibody such that an antibody component is an F(ab′) fragment or reduced IgG, the F(ab′) fragment or reduced IgG being bound with the label in a predetermined proportion, is included in the first region or an adjacent region, and
the second antibody is a biotin- or avidin-bound antibody, being of anti-idiotype antibody against the first antibody and a type that cannot bind to the bound product of the antigen and first antibody, and is included in the second region or an adjacent region.

2. The immunoassay device according to claim 1, wherein the first antibody is included in an excess amount relative to the analyte.

3. The immunoassay device according to claim 1, wherein the second antibody is included in an excess amount relative to the first antibody.

4. The immunoassay device according to claim 1, wherein the first antibody is labeled with an enzyme.

5. The immunoassay device according to claim 1, further comprising a retaining region for retaining the first antibody, which is connected to an adjacent part of the first region.

6. The immunoassay device according to claim 1, wherein a reagent for detecting the label is retained in the fourth region.

7. The immunoassay device according to claim 1, further comprising a substrate-retaining region for retaining a reagent for detecting the label, which is connected to an adjacent part of the fourth region.

8. The immunoassay device according to claim 1, wherein an electron transport mediator or chromogenic substrates retained in the substrate-retaining region.

9. The immunoassay device according to claim 1, wherein the device is in the form of a biochip, the four regions are each composed of small spaces, the immobilization means is microparticles, and the third and fourth regions are separated by a channel through which the microparticles serving as the immobilization means do not pass.

10. The immunoassay device according to claim 1, further comprising a pair of electrodes formed in the fourth region.

11. An immunoassay method comprising;
bringing about a reaction between an antigen analyte in a sample and a first antibody that is a labeled antibody specifically binding to the antigen, then
allowing an unreacted first antigen to react with a second biotin- or avidin-bound antigen, subsequently
capturing the second antibody with avidin when the second antibody is biotin-bound antibody or with biotin when the second antibody is avidin-bound antibody, and
detecting a uncaptured bound product of the antigen and the first antibody,
wherein the first antibody is a labeled antibody such that an antibody component is an F(ab′) fragment or reduced IgG, the F(ab′) fragment or reduced IgG being bound with a label in a predetermined proportion, and
the second antibody is an anti-idiotype antibody against the first antibody, being of a type that cannot bind to the bound product of the antigen and first antibody.

12. The immunoassay method according to claim 11, wherein the first antibody is used in an excess amount relative to the analyte.

13. The immunoassay method according to claim 11, wherein the second antibody is used in an excess amount relative to the first antibody.

14. The immunological analysis method according to claim 11, wherein the analyte is detected electrochemically or optically.
